SEQUENCE LISTING (10 sequences)

SEQ ID NO: 1 - DNA, 1528 bp, homo sapiens
cagtgccttggtaatgaccagggctccagaaagagatgtccttgtggctgggggcccctgtgcctgacattcctcctgactctgcggtggagctgtggaagccaggcgcacaggatgcaagcagccaggcccagggaggcagcagctgcatcctcagagaggaagccaggatgccccactctgctgggggtactgcaggggtggggctggaggctgcagagcccacagccctgctcaccagggcagagcccccttcagaacccacagagatccgtccacaaaagcggaaaaaggggccagcccccaaaatgctggggaacgagctatgcagcgtgtgtggggacaaggcctcgggcttccactacaatgttctgagctgcgagggctgcaagggattcttccgccgcagcgtcatcaagggagcgcactacatctgccacagtggcggccactgccccatggacacctacatgcgtcgcaagtgccaggagtgtcggcttcgcaaatgccgtcaggctggcatgcgggaggagtgtgtcctgtcagaagaacagatccgcctgaagaaactgaagcggcaagaggaggaacaggctcatgccacatccttgccccccaggcgttcctcacccccccaaatcctgccccagctcagcccggaacaactgggcatgatcgagaagctcgtcgctgcccagcaacagtgtaaccggcgctccttttctgaccggcttcgagtcacgccttggcccatggcaccagatccccatagccgggaggcccgtcagcagcgctttgcccacttcactgagctggccatcgtctctgtgcaggagatagttgactttgctaaacagctacccggcttcctgcagctcagccgggaggaccagattgccctgctgaagacctctgcgatcgaggtgatgcttctggagacatctcggaggtacaaccctgggagtgagagtatcaccttcctcaaggatttcagttataaccgggaagactttgccaaagcagggctgcaagtggaattcatcaaccccatcttcgagttctccagggccatgaatgagctgcaactcaatgatgccgagtttgccttgctcattgctatcagcatcttctctgcagaccggcccaacgtgcaggaccagctccaggtggagaggctgcagcacacatatgtggaagccctgcatgcctacgtctccatccaccatccccatgaccgactgatgttcccacggatgctaatgaaactggtgagcctccggaccctgagcagcgtccactcagagcaagtgtttgcactgcgtctgcaggacaaaaagctcccaccgctgctctctgagatctgggatgtgcacgaatgactgttctgtccccatattttctgttttcttggccggatggctgaggcctggtggctgcctcctagaagtggaacagactgagaagggcaaacattcctgggagctgggcaaggagatcctcccgtggcattaaaagagagtcaaagggt

SEQ ID NO: 2 - protein, 447 aa, homo sapiens
MSLWLGAPVPDIPPDSAVELWKPGAQDASSQAQGGSSCILREEARMPHSAGGTAGVGLEAAEPTALLTRAEPPSEPTEIRPQKRKKGPAPKMLGNELCSVCGDKASGFHYNVLSCEGCKGFFRRSVIKGAHYICHSGGHCPMDTYMRRKCQECRLRKCRQAGMREECVLSEEQIRLKKLKRQEEEQAHATSLPPRRSSPPQILPQLSPEQLGMIEKLVAAQQQCNRRSFSDRLRVTPWPMAPDPHSREARQQRFAHFTELAIVSVQEIVDFAKQLPGFLQLSREDQIALLKTSAIEVMLLETSRRYNPGSESITFLKDFSYNREDFAKAGLQVEFINPIFEFSRAMNELQLNDAEFALLIAISIFSADRPNVQDQLQVERLQHTYVEALHAYVSIHHPHDRLMFPRMLMKLVSLRTLSSVHSEQVFALRLQDKKLPPLLSEIWDVHE

SEQ ID NO: 3 - DNA, 2010 bp, homo sapiens
caagaagtggcgaagttacctttgagggtatttgagtagcggcggtgtgtcaggggctaaagaggaggacgaagaaaagcagagcaagggaacccagggcaacaggagtagttcactccgcgagaggccgtccacgagacccccgcgcgcaggcatgagccccgccccccacgcatgagccccgccccccgctgttgcttggagaggggcgggacctggagagaggctgctccgtgaccccaccatgtcctctcctaccacgagttccctggatacccccctgcctggaaatggcccccctcagcctggcgccccttcttcttcacccactgtaaaggaggagggtccggagccgtggcccgggggtccggaccctgatgtcccaggcactgatgaggccagctcagcctgcagcacagactgggtcatcccagatcccgaagaggaaccagagcgcaagcgaaagaagggcccagccccgaagatgctgggccacgagctttgccgtgtctgtggggacaaggcctccggcttccactacaacgtgctcagctgcgaaggctgcaagggcttcttccggcgcagtgtggtccgtggtggggccaggcgctatgcctgccggggtggcggaacctgccagatggacgctttcatgcggcgcaagtgccagcagtgccggctgcgcaagtgcaaggaggcagggatgagggagcagtgcgtcctttctgaagaacagatccggaagaagaagattcggaaacagcagcagcaggagtcacagtcacagtcgcagtcacctgtggggccgcagggcagcagcagctcagcctctgggcctggggcttcccctggtggatctgaggcaggcagccagggctccggggaaggcgagggtgtccagctaacagcggctcaagaactaatgatccagcagttggtggcggcccaactgcagtgcaacaaacgctccttctccgaccagcccaaagtcacgccctggcccctgggcgcagacccccagtcccgagatgcccgccagcaacgctttgcccacttcacggagctggccatcatctcagtccaggagatcgtggacttcgctaagcaagtgcctggtttcctgcagctgggccgggaggaccagatcgccctcctgaaggcatccactatcgagatcatgctgctagagacagccaggcgctacaaccacgagacagagtgtatcaccttcttgaaggacttcacctacagcaaggacgacttccaccgtgcaggcctgcaggtggagttcatcaaccccatcttcgagttctcgcgggccatgcggcggctgggcctggacgacgctgagtacgccctgctcatcgccatcaacatcttctcggccgaccggcccaacgtgcaggagccgggccgcgtggaggcgttgcagcagccctacgtggaggcgctgctgtcctacacgcgcatcaagaggccgcaggaccagctgcgcttcccgcgcatgctcatgaagctggtgagcctgcgcacgctgagctctgtgcactcggagcaggtcttcgccttgcggctccaggacaagaagctgccgcctctgctgtcggagatctgggacgtccacgagtgaggggctggccacccagccccacagccttgcctgaccaccctccagcagatagacgccggcaccccttcctcttcctagggtggaaggggccctgggcgagcctgtagacctatcggctctcatcccttgggataagccccagtccaggtccaggaggctccctccctgcccagcgagtcttccagaaggggtgaaagggttgcaggtcccgaccactgacccttcccggctgccctccctccccagcttacacctcaagcccagcacgcagcgtaccttgaacagagggaggggaggacccatggctctccccccctagcccgggagaccaggggccttcctcttcctctgcttttatttaataaaaataaaaacagaaa

SEQ ID NO: 4 - protein, 461 aa, homo sapiens
MSSPTTSSLDTPLPGNGPPQPGAPSSSPTVKEEGPEPWPGGPDPDVPGTDEASSACSTDWVIPDPEEEPERKRKKGPAPKMLGHELCRVCGDKASGFHYNVLSCEGCKGFFRRSVVRGGARRYACRGGGTCQMDAFMRRKCQQCRLRKCKEAGMREQCVLSEEQIRKKKIRKQQQQESQSQSQSPVGPQGSSSSASGPGASPGGSEAGSQGSGEGEGVQLTAAQELMIQQLVAAQLQCNKRSFSDQPKVTPWPLGADPQSRDARQQRFAHFTELAIISVQEIVDFAKQVPGFLQLGREDQIALLKASTIEIMLLETARRYNHETECITFLKDFTYSKDDFHRAGLQVEFINPIFEFSRAMRRLGLDDAEYALLIAINIFSADRPNVQEPGRVEALQQPYVEALLSYTRIKRPQDQLRFPRMLMKLVSLRTLSSVHSEQVFALRLQDKKLPPLLSEIWDVHE

SEQ ID NO: 5 - DNA, 5449 bp, homo sapiens
gcgccgggggccgccgcgcccgccgcccgctgcctgcgccgccggccgggcatgagttagtcgcagacatggacaccaaacatttcctgccgctcgatttctccacccaggtgaactcctccctcacctccccgacggggcgaggctccatggctgccccctcgctgcacccgtccctggggcctggcatcggctccccgggacagctgcattctcccatcagcaccctgagctcccccatcaacggcatgggcccgcctttctcggtcatcagctcccccatgggcccccactccatgtcggtgcccaccacacccaccctgggcttcagcactggcagcccccagctcagctcacctatgaaccccgtcagcagcagcgaggacatcaagccccccctgggcctcaatggcgtcctcaaggtccccgcccacccctcaggaaacatggcttccttcaccaagcacatctgcgccatctgcggggaccgctcctcaggcaagcactatggagtgtacagctgcgaggggtgcaagggcttcttcaagcggacggtgcgcaaggacctgacctacacctgccgcgacaacaaggactgcctgattgacaagcggcagcggaaccggtgccagtactgccgctaccagaagtgcctggccatgggcatgaagcgggaagccgtgcaggaggagcggcagcgtggcaaggaccggaacgagaatgaggtggagtcgaccagcagcgccaacgaggacatgccggtggagaggatcctggaggctgagctggccgtggagcccaagaccgagacctacgtggaggcaaacatggggctgaaccccagctcgccgaacgaccctgtcaccaacatttgccaagcagccgacaaacagcttttcaccctggtggagtgggccaagcggatcccacacttctcagagctgcccctggacgaccaggtcatcctgctgcgggcaggctggaatgagctgctcatcgcctccttctcccaccgctccatcgccgtgaaggacgggatcctcctggccaccgggctgcacgtccaccggaacagcgcccacagcgcaggggtgggcgccatctttgacagggtgctgacggagcttgtgtccaagatgcgggacatgcagatggacaagacggagctgggctgcctgcgcgccatcgtcctctttaaccctgactccaaggggctctcgaacccggccgaggtggaggcgctgagggagaaggtctatgcgtccttggaggcctactgcaagcacaagtacccagagcagccgggaaggttcgctaagctcttgctccgcctgccggctctgcgctccatcgggctcaaatgcctggaacatctcttcttcttcaagctcatcggggacacacccattgacaccttccttatggagatgctggaggcgccgcaccaaatgacttaggcctgcgggcccatcctttgtgcccacccgttctggccaccctgcctggacgccagctgttcttctcagcctgagccctgtccctgcccttctctgcctggcctgtttggactttggggcacagcctgtcactgctctgcctaagagatgtgttgtcaccctccttatttctgttactacttgtctgtggcccagggcagtggctttcctgaggcagcagccttcgtggcaagaactagcgtgagcccagccaggcgcctccccaccgggctctcaggacaccctgccacaccccacggggcttgggcgactacagggtcttcgggccccagccctggagctgcaggagttgggaacggggcttttgtttccgttgctgtttatcgatgctggttttcagaattcctgtgtggccctcctgtctggagtgacatcttcatctgctctgaatactggtgcccagccagcccgtgacagcttccccctaatcaggaggggacagctgggggcgcaagctggtgtgtcatcagcaaagacctcagccgcctcggggatgagaggggactcgtggggcaagcaagctgccctgtgctctgagtgagggggaaggtagcccctttttccaaagataactcacagttttgccctcgagccaatgagaacatgagctgccctctgtgcaaggtttcggggccacctccaggctgcaggggcgggtcactcacccccctgttttctctctgccttggtgttctggtttcagactcccgactccccgttcagaccagagtgccccggcccctccccagcctgagtcttctccttgctctgcggggtgggctgaggcttgtccttgtttcctgcagggctggccctggctcgggcagggtggggcatcaccacctcactggccttgctggaggcacagggctctgcggacctgcagccatctgtgaggcccgcggggatgggaggggaggagggtggcctgttggtttccctcagagggggcaggtggcctggagagagaggggctcaggaactgggagcctcgtgggtggggcagatgctccgcggcctggagtggctctgccggggcattggtgggacccctgctcaggccttctctctggctgccagttgtgtctaaaagactcttggaatctgagaacccggagtcgcagcgccctcgggcctgggccacacgcaggccctggtgggaccacccagcctggtattgtccacggacagcgttgttcacccagagccttacttgggagcctcactgaacgcctgctctggttgaaggtggggtgggggcggggcttggggcctccctggctcagcccagtgcggcctggcgctcctcccgcaggctctgcccccgggctccggtggtgcggggccctctcaggttgaactcgcctcttttgcactggaaggccctccctttggcctgagtacttttcccgttcacgcctcagtcccgtggacccagcctttgtcagtggcaggtgcctgaacagagggtggatgggggggataccggagggggtcttgtcttcccagccgcagtctaggaatgatgcgggggggtggacgccttctccatagtctttccccacctggagcaggggcttcctcagtggtgaggggagctgcctacaggttggaccgggaggcagtggcttggagaggcagctttccagccttggtggggaagaaagtgtccattctttgccttcctggagctcccagccagagctgagcttaggcacccgagtggagcctgcagctgagtctgtgcccgagacaggctgtcagagattccagaagcctctcctccccgccgccctccacccctgcctttcagcgttgtggatccctagaggtggccccctgcccgatccaccgtcctgaggcagagtgttgagcctcatacctgtaccaggtccccggccagctgggcccctcccaggcactgccaggaagccccagctgcccctggcgggtgtggtggaaatggcaggagggtgcaggtactcttggggccccagcggtgggagtgcaaaagacccaacgccaacacctggtgccttttgcagccagcgcccacccatccgtgcccggacccttgggaatgcccgcggctccagaggaaaaagcccagggacggggcctccgttgcggggggtcggctgcttcttgggaactttgtcgtttccggcgctggctggctggctggctgtaaagcactgaagccccccggccgccaacccctgaaagcagaacctggcctccctggccacagcagccttacccaccgctctacgtgtcccgggcacttcccgcagccttcccgtccctttctcatcggccttgtagttgtacagtgctgttggtttgaaaaggtgatgtgtggggagtgcggctcatcactgagtagagaggtagaatttctatttaaccagacctgtagtagtattaccaatccagttcaattaaggtgattttttgtaattattattattttggtgggacaatctttaattttctaaagatagcactaacatcagctcattagccacctgtgcctgtccccgccttggcccggctggatgaagcggcttccccgcagggcccccacttcccagtggctgcttcctggggacccagggcaccccggcaccttcaggcacgctcctcagctggtcacctcccggctttgccgttcagatggggctcctgaggctcaggagtgaagatgccacagagccgggctcccctaggctgcgtcgggcatgcttggaagctggcctgccaggaccttccaccctggggcctgtgtcagccgccggccctccgcaccctggaagcacacggcctctgggaaggacagccctgaccttcggttttccgagcacggtgtttcccaagaattctgggctggcggcctggtggcagtgctggagatgaccccgagcccctccccgtggggcacccaggagggccctgccggaatgtgcagcctgtgggtagtcggctggtgtccctgtcgtggagctggggtgcgtgatctggtgctcgtccacgcaggtgtgtggtgtaaacatgtatgtgctgtacagagagacgcgtgtggagagagccgcacaccagcgccacccaggaaaggcggagcggttaccagtgttttgtgtttatttttaatcaagacgtttcccctgttttcctataaatttgcttcgtgtaagcaagtacataaggaccctcctttggtgaaatccgggttcgaatgaatatctcaaggcaggagatgcatctattttaagatgctttggagcagacagctttagccgttcccaatccttagcaatgccttagctgggacgcatagctaatactttagagaggatgacagatccataaagagagtaaagataagagaaaatgtctaaagcatctggaaaggtaaaaaaaaaaaatctatttttgtacaaatgtaattttatccctcatgtatacttggatatggcggggggagggctgggactgtttcgtttctgcttctagagattgaggtgaaagcttcgtccgagaaacgccaggacagacgatggcagaggagagggctcctgtgacggcggcgaggcttgggaggaaaccgccgcaatgggggtgtcttccctcggggcaggagggtgggcctgaggctttcaagggttttcttccctttcgagtaatttttaaagccttgctctgttgtgtcctgttgccggctctggccttcctgtgactgactgtgaagtggcttctccgtacgattgtctctgaaacatcgtggcctcaggtgccagggtttgatggacagtagcattagaattgtggaaaaggaacacgcaaagggagaagtgtgagaggagaaacaaaatatgagcgtttaaaatacatcgccattcagttcgttaaaaaaaaaaaaaaaaaaaaaaaaa

SEQ ID NO: 6 - protein, 462 aa, homo sapiens
MDTKHFLPLDFSTQVNSSLTSPTGRGSMAAPSLHPSLGPGIGSPGQLHSPISTLSSPINGMGPPFSVISSPMGPHSMSVPTTPTLGFSTGSPQLSSPMNPVSSSEDIKPPLGLNGVLKVPAHPSGNMASFTKHICAICGDRSSGKHYGVYSCEGCKGFFKRTVRKDLTYTCRDNKDCLIDKRQRNRCQYCRYQKCLAMGMKREAVQEERQRGKDRNENEVESTSSANEDMPVERILEAELAVEPKTETYVEANMGLNPSSPNDPVTNICQAADKQLFTLVEWAKRIPHFSELPLDDQVILLRAGWNELLIASFSHRSIAVKDGILLATGLHVHRNSAHSAGVGAIFDRVLTELVSKMRDMQMDKTELGCLRAIVLFNPDSKGLSNPAEVEALREKVYASLEAYCKHKYPEQPGRFAKLLLRLPALRSIGLKCLEHLFFFKLIGDTPIDTFLMEMLEAPHQMT

SEQ ID NO: 7 - DNA, 2892 bp, homo sapiens
caaagatggctgccacattggcgctgtcattttggtactgagcagagcgacgggcttaattcgacccaatccaggccagagtctttctctcaggggcttcctcgtgctcagctaatcctccgatcaatccttgggaatccctgggacctcttcggtatccctactctcagccagggatcatgtcttgggccgctcgcccgcccttcctccctcagcggcatgccgcagggcagtgtgggccggtgggggtgcgaaaagaaatgcattgtggggtcgcgtcccggtggcggcggcgacggccctggctggatcccgcagcggcggcggcggcggcggtggcaggcggagaacaacaaaccccggagccggagccaggggaggctggacgggacgggatgggcgacagcgggcgggactcccgaagcccagacagctcctccccaaatccccttccccagggagtccctcccccttctcctcctgggccacccctacccccttcaacagctccatcccttggaggctctggggccccacccccacccccgatgccaccacccccactgggctctccctttccagtcatcagttcttccatggggtcccctggtctgccccctccagctcccccaggattctccgggcctgtcagcagcccccagattaactcaacagtgtcactccctgggggtgggtctggcccccctgaagatgtgaagccaccagtcttaggggtccggggcctgcactgtccaccccctccaggtggccctggggctggcaaacggctatgtgcaatctgcggggacagaagctcaggcaaacactacggggtttacagctgtgagggttgcaagggcttcttcaaacgcaccatccgcaaagaccttacatactcttgccgggacaacaaagactgcacagtggacaagcgccagcggaaccgctgtcagtactgccgctatcagaagtgcctggccactggcatgaagagggaggcggtacaggaggagcgtcagcggggaaaggacaaggatggggatggggagggggctgggggagcccccgaggagatgcctgtggacaggatcctggaggcagagcttgctgtggaacagaagagtgaccagggcgttgagggtcctgggggaaccgggggtagcggcagcagcccaaatgaccctgtgactaacatctgtcaggcagctgacaaacagctattcacgcttgttgagtgggcgaagaggatcccacacttttcctccttgcctctggatgatcaggtcatattgctgcgggcaggctggaatgaactcctcattgcctccttctcacaccgatccattgatgttcgagatggcatcctccttgccacaggtcttcacgtgcaccgcaactcagcccattcagcaggagtaggagccatctttgatcgggtgctgacagagctagtgtccaaaatgcgtgacatgaggatggacaagacagagcttggctgcctgagggcaatcattctgtttaatccagatgccaagggcctctccaaccctagtgaggtggaggtcctgcgggagaaagtgtatgcatcactggagacctactgcaaacagaagtaccctgagcagcagggacggtttgccaagctgctgctacgtcttcctgccctccggtccattggccttaagtgtctagagcatctgtttttcttcaagctcattggtgacacccccatcgacaccttcctcatggagatgcttgaggctccccatcaactggcctgagctcagacccagacgtggtgcttctcacactggaggagcacacatccaagagggactccaagccctggggcagggtggggggccatgttcccagaaccttgatggggtgagaagtacagggcagaaccaagaacataaaccctccaagggatctgcttgatatcccaagttggaagggaccccagatacctgtgaggactggttgtctctcttcggtggccttgagtctctgaatttgtcgggttctcccatgatttggggtgatttctcaccctctgtccttcccccagcacaaagcactggccttgcctccaggaccttgcttccttctcatcttgcctcattttgcttcccatctgaagagtggaaatggggaactcccccagaggtggatactggggggcaggcctcccaagctgatggacatgagagtagggccctgacaggccttcctcctctcaaacctggcagatgggggcctctctggaagagggaggggccctgtcactgtccagagtctctttttacacttcacctccttctgcagtcagactgaaatataaaaaaggtggtggtggtggtgaaggggctggtggagatgtaggaaccgatctgctatttttaatttcctgtgaggatagagacttgcagttagactcaaagaagtactgtactttcccaggttgactaagaaatgccagtggtggaggtgggtgtttgggaaaggcagggccctgaaatggcctgtccctagggctctccaagcactagccttcccagcttcccgccgccccccctatctcttcctgtctaacttggggaaggggcctgggctgtgaggacagggcccccacaggggatggtttcacgagtgtagtcccggaggccttccctttacagctctcctccagccctgggcacatagcataggctggggacacaggatcctggcctgagaattgaggggaggtggccagcccgcagaggtggggtgctggggctgcatgatttttgccctgcgtcccttctctttggggctcctttcccctctcatacataaaatcgctttcaaattaaaatcgctgttttctggaaaaaaaaaaaaaaaaaa

SEQ ID NO: 8 - protein, 533 aa, homo sapiens
MSWAARPPFLPQRHAAGQCGPVGVRKEMHCGVASRWRRRRPWLDPAAAAAAAVAGGEQQTPEPEPGEAGRDGMGDSGRDSRSPDSSSPNPLPQGVPPPSPPGPPLPPSTAPSLGGSGAPPPPPMPPPPLGSPFPVISSSMGSPGLPPPAPPGFSGPVSSPQINSTVSLPGGGSGPPEDVKPPVLGVRGLHCPPPPGGPGAGKRLCAICGDRSSGKHYGVYSCEGCKGFFKRTIRKDLTYSCRDNKDCTVDKRQRNRCQYCRYQKCLATGMKREAVQEERQRGKDKDGDGEGAGGAPEEMPVDRILEAELAVEQKSDQGVEGPGGTGGSGSSPNDPVTNICQAADKQLFTLVEWAKRIPHFSSLPLDDQVILLRAGWNELLIASFSHRSIDVRDGILLATGLHVHRNSAHSAGVGAIFDRVLTELVSKMRDMRMDKTELGCLRAIILFNPDAKGLSNPSEVEVLREKVYASLETYCKQKYPEQQGRFAKLLLRLPALRSIGLKCLEHLFFFKLIGDTPIDTFLMEMLEAPHQLA

SEQ ID NO: 9 - DNA, 1742 bp, homo sapiens
ctccgatctagaggcagattcctgactaatcccagagggctggcccagcctgtgctccccgggctgctaggaagcgatgaccactcttgttagcccaagttgaagaaagccgggctgtgcctgggagccgagagaggcggtaatatttagaagctgcacaggagaggaacatgaactgacgagtaaacatgtatggaaattattctcacttcatgaagtttcccgcaggctatggaggctcccctggccacactggctctacatccatgagcccatcagcagccttgtccacagggaagccaatggacagccaccccagctacacagataccccagtgagtgccccacggactctgagtgcagtggggacccccctcaatgccctgggctctccatatcgagtcatcacctctgccatgggcccaccctcaggagcacttgcagcgcctccaggaatcaacttggttgccccacccagctctcagctaaatgtggtcaacagtgtcagcagttcagaggacatcaagcccttaccagggcttcccgggattggaaacatgaactacccatccaccagccccggatctctggttaaacacatctgtgccatctgtggagacagatcctcaggaaagcactacggggtatacagttgtgaaggctgcaaagggttcttcaagaggacgataaggaaggacctcatctacacgtgtcgggataataaagactgcctcattgacaagcgtcagcgcaaccgctgccagtactgtcgctatcagaagtgccttgtcatgggcatgaagagggaagctgtgcaagaagaaagacagaggagccgagagcgagctgagagtgaggcagaatgtgctaccagtggtcatgaagacatgcctgtggagaggattctagaagctgaacttgctgttgaaccaaagacagaatcctatggtgacatgaatatggagaactcgacaaatgaccctgttaccaacatatgtcatgctgctgacaagcagcttttcaccctcgttgaatgggccaagcgtattccccacttctctgacctcaccttggaggaccaggtcattttgcttcgggcagggtggaatgaattgctgattgcctctttctcccaccgctcagtttccgtgcaggatggcatccttctggccacgggtttacatgtccaccggagcagtgcccacagtgctggggtcggctccatctttgacagagtcctaactgagctggtttccaaaatgaaagacatgcagatggacaagtcggaactgggatgcctgcgagccattgtactctttaacccagatgccaagggcctgtccaacccctctgaggtggagactctgcgagagaaggtttatgccacccttgaggcctacaccaagcagaagtatccggaacagccaggcaggtttgccaagctgctgctgcgcctcccagctctgcgttccattggcttgaaatgcctggagcacctcttcttcttcaagctcatcggggacacccccattgacaccttcctcatggagatgttggagaccccgctgcagatcacctgagccccaccagccacagcctccccacccaggatgacccctgggcaggtgtgtgtggacccccaccctgcactttcctccacctcccaccctgacccccttcctgtccccaaaatgtgatgcttataataaagaaaacctttctacaaaaaaaaaaaaaaaaaa

SEQ ID NO: 10 - protein, 463 aa, homo sapiens
MYGNYSHFMKFPAGYGGSPGHTGSTSMSPSAALSTGKPMDSHPSYTDTPVSAPRTLSAVGTPLNALGSPYRVITSAMGPPSGALAAPPGINLVAPPSSQLNVVNSVSSSEDIKPLPGLPGIGNMNYPSTSPGSLVKHICAICGDRSSGKHYGVYSCEGCKGFFKRTIRKDLIYTCRDNKDCLIDKRQRNRCQYCRYQKCLVMGMKREAVQEERQRSRERAESEAECATSGHEDMPVERILEAELAVEPKTESYGDMNMENSTNDPVTNICHAADKQLFTLVEWAKRIPHFSDLTLEDQVILLRAGWNELLIASFSHRSVSVQDGILLATGLHVHRSSAHSAGVGSIFDRVLTELVSKMKDMQMDKSELGCLRAIVLFNPDAKGLSNPSEVETLREKVYATLEAYTKQKYPEQPGRFAKLLLRLPALRSIGLKCLEHLFFFKLIGDTPIDTFLMEMLETPLQIT

(19) United States
(12) Patent Application Publication
(10) Pub. No.: US 2005/0123580 A1
(43) Pub. Date: Jun. 9, 2005

(54) Method of treating atherosclerosis and hypercholesterolemia

(76) Inventor: Thomas Patrick Burris, Nobleville, IN (US)

Correspondence Address: ELI LILLY AND COMPANY;PATENT DIVISION, P.O. BOX 6288, INDIANAPOLIS, IN, 46206-6288, US

(21) Appl. No.: 10/500,912

(22) PCT Filed: Feb. 14, 2003
(86) PCT No.: PCT/US03/02685

Related U.S. Application Data
(60) Provisional application No. 60/361,383, filed on Feb. 28, 2002.

(51) Int. Cl. A61K 31/203; A61F 2/00

(52) U.S. Cl. 424/423; 514/559

(57) ABSTRACT

A method of treatment of cardiovascular diseases comprising the use of an LXR agonist impregnated on a stent is disclosed.

### FIELD OF THE INVENTION

This invention relates to a novel method for the treatment of atherosclerosis and/or hypercholesterolemia.

### BACKGROUND OF THE INVENTION

Cardiovascular Disease is a leading cause of death and disability among most of the world's population but particularly in developed and developing countries. Atherosclerosis is one of the more common forms of cardiovascular disease and it leads to insufficient blood supply to critical body organs resulting in for example, heart attack, stroke and kidney failure. Atherosclerosis also causes complications in people suffering from hypertension and diabetes.

While the processes causing atherosclerosis are complex and not completely understood, an underlying pathology to the numerous theories for the cause of atherosclerosis and atherosclerotic lesion formation are for example, an increase in serum cholesterol, and the accumulation of cholesterol esters in the arterial wall. A similar pathology is also implicated in restenosis, the so-called recurrence of stenosis or arterial stricture after corrective surgery. Restenosis has been described as an accelerated atherosclerosis induced by injury (Forrester, J. S., et al., JACC, 17(3):758-769 (1991)).

Restenosis has been observed to occur after coronary artery bypass surgery, heart transplantation, atherectomy, laser ablation, and balloon angioplasty. Restenosis is most common after balloon angioplasty; also referred to as percutaneous transluminal coronary angioplasty, which is widely used as a treatment modality in patients with coronary artery disease to reduce lumen obstruction and improve coronary blood, flow. It is estimated that between 25-35% of patients develop restenosis within 1-3 months after balloon coronary angioplasty, necessitating further interventions such as repeat angioplasty or coronary bypass surgery.

Oxysterol (LXR) receptors have been found to mediate inhibition of cholesterol absorption (uptake) and promote cholesterol efflux in the artery indicating that compounds activating LXR may be used as therapies to treat restenosis. LXR receptors in combination with retinoid (X) receptors (RXR) serve as regulators of cholesterol balance by controlling reverse cholesterol transport from peripheral tissues, bile acid synthesis in the liver, and cholesterol absorption in the intestine (see Mangelsdorf et al., Regulation of Absorption and ABC-1 Mediated Efflux of Cholesterol by RXR Heterodimers, Research Articles:published May 31, 2000.

LXR and RXR agonists have also been shown to have a detrimental effect on plasma triglyceride levels via direct action at the liver (Schultz et al., Role of LXRs in Control of Lypogenesis, Genes and Development, 14:2831-7400). A method is therefore needed that advantageously utilizes the beneficial effects of LXR agonists while avoiding or minimizing the detrimental effects on plasma triglycerides. However, none of the available methods has been found to be sufficiently effective in lowering cholesterol absorption in a sustained manner to the atherosclerotic lesion and also limit, minimize, or ameliorate the detrimental effect on triglyceride levels via direct action in the liver.

### SUMMARY OF THE INVENTION

The present invention provides a method for lowering cholesterol absorption in a sustained manner by the use of a localized delivery of LXR and/or RXR agonists while also limiting, minimizing, or ameliorating the detrimental effect of LXR agonists on triglyceride levels via direct action in the liver.

The present invention relates to a method for localized delivery of LXR, and/or RXR ligands to atherosclerotic lesions to reduce the incidence of restinosis.

The present invention provides methods for the localized delivery of LXR and/or RXR agents to atherosclerotic lesions via catherization techniques.

The present invention provides a method for treating stroke and/or preventing restenosis by using an LXR agonist impregnated on a stent to keep the arteries open and simultaneously elevate HDL levels.

The present invention also relates to the use of a combination therapy of LXR agonist, RXR agonist and stent for the treatment and/or prevention of Cardiovascular Diseases.

The present invention relates to the use of a pharmaceutical composition comprising a therapeutically effective amount of an LXR agonist impregnated on a stent for the manufacture of a medicament for the treatment and/or prevention of Cardiovascular Diseases.

The present invention relates to the use of a pharmaceutical composition comprising a therapeutically effective amount of a combination of LXR agonist and RXR agonist impregnated on a stent for the manufacture of a medicament for the treatment and/or prevention of Cardiovascular Diseases.

### II. DEFINITIONS

The terms, “mammal” and “mammalian” include human and domesticated quadrupeds.

The term, “Cardiovascular Diseases” refers to diseases such as coronary occlusion, congestive heart failure, cardiac alternation, ventricular aneurysm, mural aneurysm, myocardial infarction, cardiac arrest, cardiac dysrhythmia, cardiac edema, cardiac dyspnea, cardiac failure, tachycardia, cardiac hemoptysis, cardiac incompetence, cardiac murmur, cardiac syncope, cardiac tamponade.

The term “hypercholesterolemia” refers to an abnormally large amount of cholesterol present in the cells and/or plasma of circulating blood.

The term “antihypercholesterolemic agent” refers to agents that inhibit cholesterol absorption i.e. liver X receptor (LXR) ligands for the potential treatment of hypercholesterolemia. Such inhibitors include for example, LXR agonists, derivatives and analogs of LXR ligands such as T-0901317 and TU-314407, as well as derivatives and analogs of hydroxy-substituted azetidone compounds such as ezemtimibe (Sch-58235) and Sch-60663.

“Administering” as used herein is intended to include routes of administration, which allow the antihypercholesterolemic agent to perform its intended function of lowering cholesterol absorption. Such administration includes systemic and local or site specific administration by means of a drug delivery catheter, or implantation of a drug-carrying device.

The term “treatment” as used herein refers to the amelioration, inhibition, prevention of recurrence, reduction in severity or effect, of cardiovascular diseases including but not limited to hypercholesterolemia, and artheroscierosis by the use of a stent impregnated with LXR ligand(s) and/or RXR ligands.

The term “effective amount” as used herein refers to the amount of LXR ligand(s) and/or RXR ligands necessary or sufficient to lower the absorption of cholesterol in the atherosclerotic lesion and/or elevate the level of HDL. The effective amount can vary depending on factors known to those of skill in the art, such as mode and regimen of administration, the size of the subject, severity of hypercholesterolemia, etc. One of skill in the art would be able to consider such factors and make the determination regarding effective amount.

“Pharmaceutically acceptable carrier” refers to any substance co-administered with the antihypercholesterolemic agent and which allows the compound to perform its intended function. Examples of such carriers include solutions, solvents, dispersion media, delay agents, emulsions, microparticles and the like for combination therapies.

The term, “alkyl” by itself or as part of another substituent means, unless otherwise defined, a straight or branched chain monovalent hydrocarbon radical such as methyl, ethyl, n-propyl, isopropyl, n-butyl, tertiary butyl, sec-butyl, n-pentyl, and n-hexyl.

The term, “alkenyl” employed alone or in combination with other terms means a straight chain or branched monovalent hydrocarbon group having the stated number ranges of carbon atoms, and typified by groups such as vinyl, propenyl, crotonyl, isopentenyl, and various butenyl isomers.

The term, “hydrocarbyl” means an organic group containing only carbon and hydrogen.

The term, “halo” means fluoro, chloro, bromo, or iodo.

The term “heterocyclic radical” refers to radicals derived from monocyclic or polycyclic, saturated or unsaturated, substituted or unsubstituted heterocyclic nuclei having 5 to 14 ring atoms and containing from 1 to 3 hetero atoms selected from the group consisting of nitrogen, oxygen or sulfur. Typical heterocyclic radicals are pyrrolyl, pyrrolodinyl, piperidinyl, furanyl, thiophenyl, pyrazolyl, imidazolyl, phenylimidazolyl, triazolyl, isoxazolyl, oxazolyl, thiazolyl, thiadiazolyl, benzo(b)thiophenyl, carbazolyl, norharmanyl, azabenzo(b)thiophenyl, benzofuranyl, dibenzofuranyl, dibenzothiophenyl, indazolyl, imidazo(1.2-A)pyridinyl, benzotriazolyl, anthranilyl, 1,2-benzisoxazolyl, benzoxazolyl, benzothiazolyl, purinyl, pyridinyl, dipyridylyl. phenylpyridinyl, benzylpyridinyl, pyrimidinyl, phenylpyrimidinyl, pyrazinyl, 1,3,5-triazinyl, quinolinyl, phihalazinyl, quinazolinyl,morpholino, thiomorpholino, homopiperazinyl, tetrahydrofuranyl, tetrahydropyranyl, oxacanyl, 1,3-dioxolanyl, 1,3-dioxanyl, 1,4-dioxanyl, tetrahydrothiophenyl, pentamethylenesulfadyl, 1,3-dithianyl, 1,4-dithianyl, 1,4-thioxanyl, azetidinyl, hexamethyleneiminium, heptamethyleneiminium, piperazinyl and quinoxalinyl.

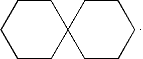

### III. THE LXR AGONISTS OF THE INVENTION

One embodiment of the practice of the present invention is the use of a pharmaceutical composition comprising a therapeutically effective amount of an LXR agonist of formula I impregnated on a stent for the treatment and/or prevention of Cardiovascular Diseases 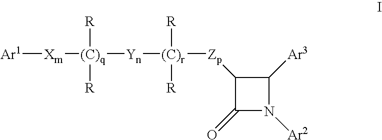
or a pharmaceutically acceptable salt thereof, wherein:
Ar¹ and Ar² are independently selected from the group consisting of aryl and R⁴-substituted aryl;
Ar³ is aryl or R⁵-substituted aryl;
X, Y and Z are independently selected from the group consisting of —CH₂—, —CH(lower alkyl)- and
—C(dilower alkyl)-;
R and R² are independently selected from the group consisting of —OR⁶, —O(CO)R⁶, —O(CO)OR⁹ and —O(CO)NR⁶R⁷;
R¹ and R³ are independently selected from the group consisting of hydrogen, lower alkyl and aryl;
q is 0 or 1; r is 0 or 1; m, n and p are independently 0, 1, 2, 3 or 4; provided that at least one of q and r is 1, and the sum of m, n, p, q and r is 1, 2, 3, 4, 5 or 6; and provided that when p is 0 and r is 1, the sum of m, q and n is 1, 2, 3, 4 or 5;
R⁴ is 1-5 substituents independently selected from the group consisting of lower alkyl, —OR⁶, —O(CO)R⁶, —O(CO)OR⁹, —O(CH₂)₁₋₅OR⁶, —O(CO)NR⁶R⁷, —NR⁶R⁷. —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, S(O)₀₋₂R⁹, —O(CH₂)₁₋₁₀—COOR⁶, —O(CH₂)₁₋₁₀CONR⁶R⁷, -(lower alkylene)COOR⁶, —CH═CH—COOR⁶, —CF₃, —CN, —NO₂ and halogen;
R⁵ is 1-5 substituents independently selected from the group consisting of —OR⁶, —O(CO)R⁶, —O(CO)OR⁹, —O(CH₂)₁₋₅OR⁶, —O(CO)NR⁶R⁷, —NR⁶R⁷, —NR⁶(CO)R⁷, —NR⁶(CO)OR⁹, —NR⁶(CO)NR⁷R⁸, —NR⁶SO₂R⁹, —COOR⁶, —CONR⁶R⁷, —COR⁶, —SO₂NR⁶R⁷, —S(O)₀₋₂R⁹, —O(CH₂)₁₋₁₀—COOR⁶, —O(CH₂)₁₋₁₀CONR⁶R⁷, -(lower alkylene)COOR⁶, —CH=CH—COOR⁶;
R⁶ R⁷ AND R⁸ are independently selected from the group consisting of hydrogen, lower alkyl, aryl and aryl-substituted lower alkyl; and
R⁹ is lower alkyl, aryl or aryl-substituted lower alkyl.

Another embodiment of the practice of the present invention is the use of a pharmaceutical composition comprising a therapeutically effective amount of an LXR agonist of formula II impregnated on a stent for the treatment and/or prevention of Cardiovascular Diseases wherein formula II is represented by the: 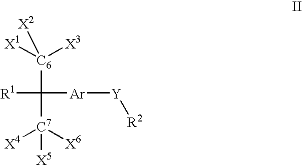
a pharmaceutically acceptable salt thereof, wherein
Ar is an aryl group;
R¹ is a member selected from the group consisting of —OH,—CO₂H, —O—(C₁-C₇)alkyl, —OC(O)—(C₁-C₇)alkyl, —O—(C₁-C₇)heteroalkyl, —OC(O)—(C₁-C₇)heteroalkyl, —NH₂,—NH(C₁-C₇)alkyl, —N((C₁-C₇)alkyl)₂ and —NH—S(O)₂—(C₁-C₅)alkyl;
R² is a member selected from the group consisting of (C₁-C₇)alkyl or (C₁-C₇)heteroalkyl, aryl and aryl(C₁-C₇)alkyl;
X¹, X², X³, X⁴, X⁵ and X⁶ are each independently a member selected from the group consisting of H,(C₁-C₅)heteroalkyl, F and Cl, with the proviso that no more than three of X¹ through X⁶ are H, (C₁-C₅)alkyl or (C₁-C₇)heteroalkyl; and
Y is a divalent linking group selected from the group consisting of —N(R¹²)S(O)ₘ—, —N(R¹²)S(O)ₘN(R¹³)—, —N(R¹²)C(O)—, —N(R¹²)C(O)N(R¹³)—, —N(R¹²)C(S)— and —N(R¹²)C(O)O—;
Wherein R¹² and R¹³ are each independently selected from the group consisting of H, (C₁-C₇)alkyl, (C₁-C₇)heteroalkyl, aryl and aryl(C₁-C₇)alkyl, and optionally when Y is —N(R¹²)S(O)ₘ— or —N(R²)S(O)ₘN(R¹³)—, R¹² forms a five- or six-member ring fused to Ar or to R² through covalent attachment to Ar or to R² through covalent attachment to Ar or to R², respectively; and the subscript m is an integer of from 1 to 2;
With the proviso that when R¹ is OH, and —Y—R is —N(R¹²)S(O)ₘ—R² or —N(R¹²)C(O)N(R¹³)—R² and is attached to a position para to the quaternary carbon attached to Ar, and when R² is phenyl, benzyl or benzoyl, then i) at least one of R¹² or R¹³ is other than hydrogen and contains an electron-withdrawing substitutent, or ii) R² is substituted with a moiety other than amino, acetamido, di(C₁-C₇)alkylamino, 1(C₁-C₇)alkylamino, halogen, hydroxy, nitro, or (C₁-C₇)alkyl, or iii) the benzene ring portion of R² is substituted with at least three independently selected groups in addition to the Y group or point of attachment to Y. Compounds of formula H are disclosed in PCT application number PCT/US00/06611, filed Mar. 15, 2000, of which the method of preparation and examples are incorporated herein.

Methods and procedures for preparing compounds of formula I are disclosed in PCT application Number PCT/US94/10099, filed Sep. 14,1994, and are incorporated herein by reference.

Methods and procedures for preparing compounds of formula II are disclosed in PCT application number PCT/US00/06611, filed Mar. 15, 2000, and are incorporated herein by reference.

A compound of formula I and/or formula II along with a carrier and or excipients is impregnated on a stent by impregnation methods known to one of skill in the art, including for example, spray-on techniques with or without pharmaceutically acceptable adhesion agents. The stent may also be immersed in a slurry or solution of the Active ingredient in a suitable solvent, i.e. methylene chloride or acetone followed by evaporation or concentration of the solution or solvent to effect impregnation of the Active ingredient on the stent. The impregnated stent may be further dried, annealed or sealed with a sealing agent to prevent flaking off or break-offs. Annealing and/or sealing agents for the purpose are known to one of skill in the art.

### IV. METHODS OF USING THE INVENTION

The LXR agonist/stent or LXR/RXR agonist/stent combinations described herein are believed to achieve their beneficial therapeutic action by simultaneously providing stenting action and cholesterol efflux, and thereby treating and/or preventing artherosclerosis and restenosis.

The method of the invention for inhibiting restenosis and effecting cholesterol efflux comprises contacting arterial cavity with a therapeutically effective amount of an LXR agonist or a combination of an LXR agonist and an RXR agonist adsorbed on, or impregnated on a stent as described herein including a salt or a prodrug derivative of LXR and/or RXR agonist thereof.

Another aspect of this invention relates to a method for treating Cardiovascular Diseases such as coronary occlusion, congestive heart failure, cardiac alternation, ventricular aneurysm, mural aneurysm, myocardial infarction, cardiac arrest, cardiac dysrhythmia, cardiac edema, cardiac dyspnea, cardiac failure, tachycardia, cardiac hemoptysis, cardiac incompetence, cardiac murmur, cardiac syncope, cardiac tamponade.

As noted previously, the compounds useful in this invention inhibit cholesterol absorption or resorption. By the term, “inhibiting” is meant the prevention or therapeutically significant reduction in the level of cholesterol and/or prevention or therapeutically significant reduction in the risk of restenosis.

The specific dose of a compound administered according to this invention to obtain therapeutic or prophylactic effect will, of course, be determined by the particular circumstances surrounding the case, including, for example, the compound administered, the route of administration and the condition being treated. Typical daily doses will contain a non-toxic dosage level of from about 0.01 mg/kg to about 50 mg/kg of body weight of an active compound of this invention.

The LXR agonist, RXR agonist, or LXR/R combination agonist compound(s) impregnated on a stent may be administered directly at the artherosclerotic lesion via a catetherization technique. When the LXR agonist is impregnated on a stent, the dose is a factor of 2 to 20 times higher than a single therapy, single dose formulation. In the cases where the LXR agonist compound is impregnated on a stent, a slow release formulation of LXR agonist compound is applied to effect slow and timed release of formulation comprising the compound.

Pharmaceutical formulations of the invention are prepared by impregnating e.g., by spray-on, a therapeutically effective amount of the LXR agonist, RXR agonist, or LXR/RXR combination agonist compound(s) on a stent device. The spray-on pharmaceutical formulations are prepared by known procedures using known and readily available ingredients.

For the pharmaceutical formulations any suitable carrier known in the art can be used. In such a formulation, the carrier may be a solid, liquid, or mixture of a solid and a liquid. For example, the Active ingredient may be dissolved in a suitable solvent at a concentration of about 2 to 200 mg/ml in a 4% dextrose/0.5% Na citrate aqueous solution. Solid form formulations for impregnation on the stent include powders and pastes. A solid carrier can be one or more substance, which may also act as lubricants, solubilizers, suspending agents, and pharmaceutically acceptable adhesive agents.

In powders, the carrier is a finely divided solid having the necessary binding properties in suitable proportions, which is in an admixture with the finely divided Active ingredient. The powders will typically be sprayed on optionally followed by spray-on of annealing or sealing agents. The powders preferably contain from about 1 to about 99 weight percent of the Active ingredient. Suitable solid carriers are magnesium carbonate, magnesium stearate, talc, sugar lactose, pectin, dextrin, starch, gelatin, tragacanth, methylcellulose, sodium carboxymethyl cellulose, pharmaceutically acceptable low melting waxes, and pharmaceutically acceptable adhesives.

The Active ingredient can be dissolved or suspended in a pharmaceutically acceptable carrier, such as sterile water, sterile organic solvent or a mixture of both. The Active ingredient can often be dissolved in a suitable organic solvent, for instance aqueous propylene glycol. Dispersing the finely divided Active ingredient in aqueous starch or sodium carboxymethyl cellulose solution, or binder or pharmaceutically acceptable adhesive may result in other compositions. The solution or suspension is then impregnated on a stent by coating the admixture of active ingredient on the stent and allowing the solvent to evaporate slowly under vacuum until nearly all solvent or liquid is evaporated.

The following pharmaceutical formulations are illustrative only and are not intended to limit the scope of the invention in any way. “Active ingredient”, refers to a compound according to Formula (I) or (II) or a pharmaceutically acceptable salt, solvate, or prodrug thereof which is to be impregnated on a stent.

Slow Release Formulation 1
Hard gelatin powder to be sprayed on a stent is
prepared using the following ingredients:

| | Quantity |
|---|---|
| | (mg/capsule) |
| Active ingredient | 250 |
| Starch, dried | 200 |
| Magnesium stearate | 10 |
| Total | 460 mg |

Formulation 2
A solid composition of formula I or II to be sprayed
on a stent is prepared using the ingredients below:

| | Quantity |
|---|---|
| | (mg/tablet) |
| Active ingredient | 250 |
| Cellulose, microcrystalline | 400 |
| Silicon dioxide, fumed | 10 |
| Stearic acid | 5 |
| Total | 665 mg |

The components are blended and compressed to form a solid each weighing 665 mg which is then sprayed on the stent either as a slurry or admixed with a pharmaceutically acceptable adhesion agent.

1. A method for lowering cholesterol absorption in a sustained manner by the use of a localized delivery of LXR and/or RXR agonists while also limiting, minimizing, or ameliorating the detrimental effect of LXR agonists on triglyceride levels via direct action in the liver.

2. A method for treating and/or preventing Cardiovascular Disease comprising impregnation of LXR agonists on a stent device.

3. A method for treating and/or preventing Cardiovascular Diseases comprising impregnation of RXR agonists on a stent device.

4. A method for treating and/or preventing Cardiovascular Diseases comprising impregnation of LXR and/or RXR agonists on a stent device.

5. A method according to claim 1 or 3 wherein the LXR agonist is selected from a group consisting of: a compound of formula I, and a compound of formula II.

6. A composition comprising a therapeutically effective amount of a LXR agonist impregnated on a stent.

7. A composition comprising a therapeutically effective amount of a RXR agonist impregnated on a stent.

8. A composition according to claim 6 or 7 wherein the LXR or RXR agonist is impregnated to effect a time-release (slow-release) formulation.

9. (canceled)

10. (canceled)
